(19) United States
(12) Patent Application Publication
(10) Pub. No.: US 2012/0190637 A1
(43) Pub. Date: Jul. 26, 2012

(54) COMBINATION THERAPY TREATMENT FOR VIRAL INFECTIONS

(75) Inventors: William J. Guilford, Belmont, CA (US); Daryl H. Faulds, Millbrae, CA (US)

(73) Assignee: GEMMUS PHARMA, INC., San Francisco, CA (US)

(21) Appl. No.: 13/129,108

(22) PCT Filed: Oct. 13, 2010
(86) PCT No.: PCT/US10/52506

§ 371 Date: Jul. 20, 2011

Related U.S. Application Data
(60) Provisional application No. 61/251,561, filed on Oct. 14, 2009.

(51) Int. Cl. A61K 31/343; A61K 31/56; A61P 31/16; A61P 31/14; A61K 31/7056; A61K 31/215

(52) U.S. Cl. 514/43; 514/468; 514/529; 514/171

(57) ABSTRACT

Therapeutics which employ a combination of an antiviral agent and an EP4 receptor agonist for the treatment of human respiratory diseases associated with viral infections are described. Viral infections may include an influenza A virus, for example H1N1, H3N2 and H5N1, and mutations thereof, and/or a coronavirus, for example a virus that causes severe acute respiratory syndrome, “SARS”.

### CROSS-REFERENCE TO RELATED APPLICATIONS

This application claims the benefit of and priority to U.S. Provisional Application Ser. No. 61/251,561 filed Oct. 14, 2009, the contents of which are incorporated herein by reference in their entirety and for all purposes.

### FIELD

The present invention is related to combination therapies for viral infections. More particularly, combination therapies that employ one or more antiviral agents with one or more prostaglandin receptor agonists.

### BACKGROUND

A virus is an infectious agent that is identified using the Baltimore classification method based on their genetic material, DNA or RNA, and their protective coat. Since viruses are unable to reproduce on their own, they infect plant or animal cells and redirect cellular activities to the production of viral particles.

Plants and animals have devised elaborate mechanisms to fight off viral infections. In humans this defense mechanism is characterized by an immediate innate immune response which is followed by an adaptive immune response. The innate immune response is a rapid, non-selective attack on any foreign organism compared to the specific adaptive response which targets the invading organism. The success of a viral infection depends on the virus's ability to elude rapid elimination by the host's immune system. Specific cases which are responsible for human diseases of the respiratory tract include influenza virus and coronavirus that cause severe acute respiratory syndrome (SARS).

The disease state associated with viral infections is the result of tissue damage from the direct lysis of infected cells (for example, see MD de Jong et al., N. Engl. J. Med. 2005 352:686-691). To counter a viral infection, the human immune system will respond by increasing the production of pro-inflammatory cytokines. However, when cytokine production becomes prolonged or excessive it can, for example, inflame airways, making it hard to breathe, which in turn can result in pneumonia and acute respiratory distress. The excessive production of pro-inflammatory cytokines is described as a “cytokine storm” (see, for example, C W Chan, et al., Respiratory Research 2005, 6:135; MD de Jong, et al. Nat Med 2006 12:1203-1207). Prolonged or excessive cytokine production can also injure other organs, which can result in severe life-threatening complications. As one example, the severity and high morbidity and mortality associated with the Influenza A subtype H5N1 infection in humans is characterized by high viral load and hypercytokinemia. As a second example, the severity associated with seasonal influenza A infections in humans has been correlated to the hypercytokinemia (see, for example, M L Heltzer, et al., J. Leukoc. Biol. 2009; 85(6):1036-1043).

### SUMMARY

Therapeutics which employ a combination of an antiviral agent and an EP4 receptor agonist for the treatment of human respiratory diseases associated with viral infections are described. Viral infections may include an influenza A virus, for example H1N1, H3N2 and H5N1, and mutations thereof, and/or a coronavirus, for example a virus that causes severe acute respiratory syndrome, “SARS”. Methods of treating diseases associated with influenza A virus infection and/or diseases associated with a coronavirus infection are described. Methods of treating respiratory viral infections are described.

In certain embodiments, methods include administering to a patient in need thereof at least one antiviral agent and at least one EP4 receptor agonist in a synergistic combination such that their combined effect is greater than the sum of their individual effects in treating a viral disease and an associated cytokine storm. In certain embodiments, a single antiviral agent is administered in combination with a single EP4 agonist in order to achieve the synergistic therapeutic effect. An anti-inflammatory, analgesic, PPAR-γ agonist and/or immune response modulator may be added to the combination.

One embodiment is a combination of an EP4 receptor agonist and an antiviral for use in the treatment of an infection in a human by an influenza virus and/or a corona virus. In one embodiment, at least one of the EP4 receptor agonist and the antiviral agent in the combination are suboptimal dosages as described herein. In one embodiment, the antiviral agent is selected from an antiviral described herein. In one embodiment, the EP4 agonist is selected from those described herein. The combination can be administered adjunctively with an anti-inflammatory agent as described herein. The combination can also be administered with an analgesic as described herein. One embodiment is a pharmaceutical composition used according to any method of treatment described or claimed herein. One embodiment is a combination of an EP4 receptor agonist and an antiviral used according to any method of treatment described or claimed herein.

These and other features and advantages are further discussed below with reference to the associated drawings.

### DETAILED DESCRIPTION

Treatments with antiviral agents that focus exclusively on the virus, for example, neuroaminidase inhibitors such as oseltamivir, zanamivir or peramivir; M2 channel inhibitors such as amantadine and rimantadine; or the polymerase inhibitor T-705, are able to reduce viral load but do not act to prevent the release of pro-inflammatory cytokines or the resulting tissue damage caused by them. Also, antiviral agents can be rendered ineffective through induced or random viral mutations. At the same time, treatments that focus exclusively on reducing the cytokine storm, for example EP4 receptor agonists such as nileprost, beraprost, cicaprost, eptaloprost, ciprosten, enprostil, CP-533536, rivenprost, ONO-8815Ly, nocloprost, and AGN-205203, are unable to directly reduce the high viral load or stop viral induction of the cytokine storm. For example, standard steroidal anti-inflammatory therapy against avian flu has been of little therapeutic value, because steroids typically inhibit the immune system. Thus, reduction of either viral loads or the cytokine storm results in only a partial treatment of viral infections.

Surprisingly, it has been discovered that a combination of an EP4 receptor agonist and certain antiviral agents work synergistically to treat viral diseases that induce a cytokine storm. That is, treatment of a viral disease with an EP4 receptor agonist in combination with an antiviral agent results in significant, greater-than-additive increases in survival compared to treatment with either drug alone. The EP4 receptor agonist is coadministered with the antiviral agent, where sub-optimal doses of one or both agents are used. In one embodiment, an anti-inflammatory compound may also be coadministered with the EP4 receptor agonist and the antiviral agent. In one embodiment, the anti-inflammatory is a non-steroidal anti-inflammatory.

### Viral Infections

Methods described herein are used to treat viral infection. Of particular import are viral infections that induce increased production of pro-inflammatory cytokines, including such induction that rises to the level of a cytokine storm. In one embodiment, the viral infection is caused by an influenza virus, such as, but not limited to, the subtypes H1N1, H3N2, and H5N1, and their variants. In one embodiment, the viral infection is caused by a coronavirus, such as a virus that causes severe acute respiratory syndrome (SARS).

### EP4 Receptor Agonists

EP4 receptor agonists useful for carrying out methods described herein include all those that inhibit the release of cytokines and/or chemokines in response to a viral infection that induces overproduction of pro-inflammatory cytokines, including inducement that rises to the level of a cytokine storm.

In one embodiment, the EP4 receptor agonist is selected from 5-cyano-prostacyclin derivatives. Such 5-cyano-prostacyclin derivatives, their pharmacological effects as well as synthesis of the compounds and pharmaceutically acceptable salts thereof are reported in U.S. Pat. Nos. 4,219,479, 4,049,582 and 7,776,896, each of which is incorporated by reference herein for all purposes. Cyclodextrin clathrates of 5-cyano-prostacyclin derivatives are also included within the scope of EP4 agonists described herein. Such cyclodextrin clathrates are described in U.S. Pat. No. 5,010,065, which is incorporated by reference herein for all purposes.

In one embodiment, the EP4 receptor agonist is selected from certain prostacyclin and carbacyclin derivatives that are disclosed, together with methods for their synthesis in the following patents: U.S. Pat. Nos. 4,423,067, 4,474,802, 4,692,464, 4,708,963, 5,013,758; European patent EP0084856 and Canadian patent CA 1248525, each of which is incorporated by reference herein for all purposes.

In another embodiment, the EP4 receptor agonist is selected from a compound described in one of the following patents or patent application publications: U.S. Pat. No. 6,747,037, WO 20044071428, US20040102499, US2005049227, US2005228185, US2006106088, WO2006052630, WO2006047476, US2006111430, WO2006058080, US20070010495, US20070123568, US20070123569, U.S. Pat. No. 7,776,896, WO 2004065365, US20050020686, US20080234337, US20100010222, US20100216689, WO 03/047513, WO 2004085421, WO2004085430, WO2005116010, WO2005116010, WO2007014454, US20040198701, US20040204590, US20050227969, US20050239872, US20060154899, US20060167081, US20060258726, US20060270721, US20090105234, US20090105321, US20090247596, US20090258918, US20090270395), WO2006080323, US20040087624, US20040102508, US20060252799, US20090030061, US20090170931, US20100022650, US20090312388, US20090318523, US20100069457, US20100076048, WO06137472, US20070066618, US20040259921, US20050065133 and US20070191319.

In one embodiment, the EP4 receptor agonist is selected from the group consisting of (E)-4-(2-hydroxy-1-(3-hydroxy-4-methyloct-1-en-6-ynyl)-2,3,3a,8b-tetrahydro-1H-benzo[d]cyclopenta[b]furan-5-yl)butanoic acid, (E)-5-cyano-5-(5-hydroxy-4-((E)-3-hydroxy-4-methyloct-1-enyl)hexahydro-2H-cyclopenta[b]furan-2-ylidene)pentanoic acid, (E)-2-(5-hydroxy-4-((E)-3-hydroxy-4-methyloct-1-enyl)hexahydro-2H-cyclopenta[b]furan-2-ylidene)-5-(1H-tetrazol-5-yl)pentanenitrile, (E)-7-(3-hydroxy-2-(3-hydroxy-4-phenoxybut-1-enyl)-5-oxocyclopentyl)hepta-4,5-dienoic acid, (Z)-7-(5-chloro-3-hydroxy-2-((E)-3-hydroxy-4,4-dimethyloct-1-enyl)cyclopentyl)hept-5-enoic acid, (Z)-7-(3-hydroxy-2-((E)-3-hydroxy-3-methyloct-1-enyl)-5-oxocyclopentyl)hept-5-enoic acid, 2-(3-((N-(4-tert-butylbenzyl)pyridine-3-sulfonamido)methyl)phenoxy)acetic acid, (E)-4-(2-(3-hydroxy-2-(3-hydroxy-4-(3-(methoxymethyl)phenyl)but-1-enyl)-5-oxocyclopentyl)ethylthio)butanoic acid, (E)-2-(3-(3-hydroxy-2-(3-hydroxy-4-(3-(methoxymethyl)phenyl)but-1-enyl)-5-oxocyclopentylthio)propylthio)acetic acid, 4-(2-(2-((1Z,3E)-4-methylocta-1,3-dienyl)-5-oxopyrrolidin-1-yl)ethyl)benzoic acid, (E)-7-(2-(3-hydroxy-4-phenylbut-1-enyl)-6-oxopiperidin-1-yl)heptanoic acid, (E)-1-(6-(1H-tetrazol-5-yl)hexyl)-5-(4,4-difluoro-3-hydroxy-4-phenylbut-1-enyl)pyrrolidin-2-one, (E)-4-(2-(5-hydroxy-4-(3-hydroxy-4-methylnona-1,6-diynyl)hexahydropentalen-2(1H)-ylidene)ethoxy)butanoic acid and pharmaceutically acceptable salts thereof. Also included are C₁₋₆alkyl esters of any of the aforementioned carboxylic acids.

In one embodiment, the EP4 agonist is selected from a compound in Table 1:

TABLE 1

| | |
|---|---|
| 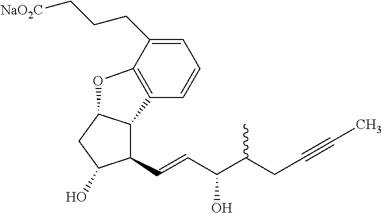 | (1) |
| 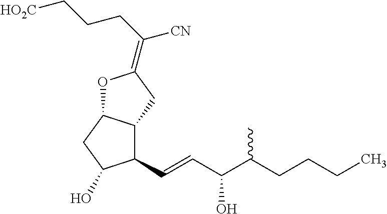 | (2) |
| 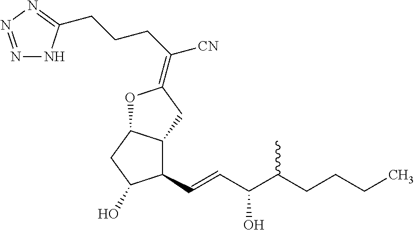 | (3) |
| 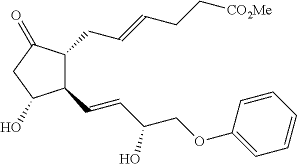 | (4) |
| 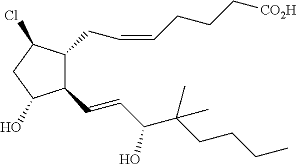 | (5) |
| 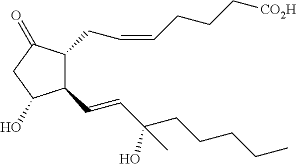 | (6) |
| 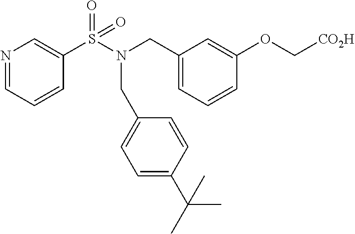 | (7) |
| 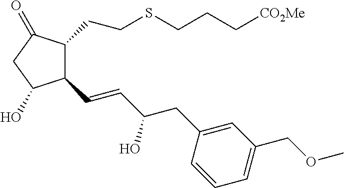 | (8) |
| 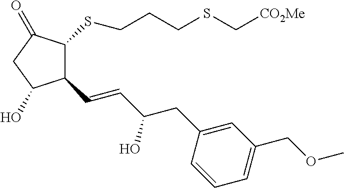 | (9) |
| 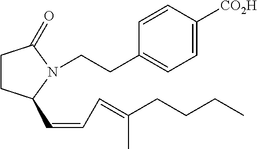 | (10) |
| 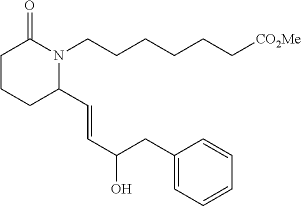 | (11) |
| 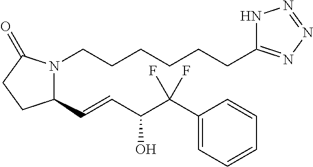 | (12) |
| 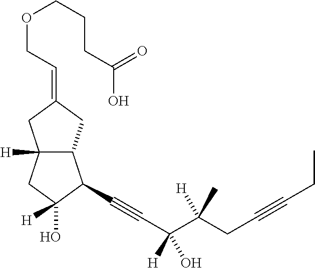 | (13) |
| 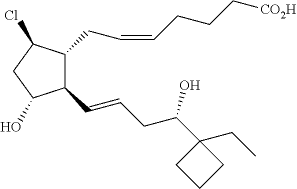 | (14) |
| 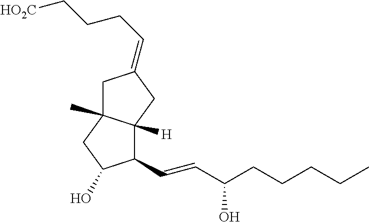 | (15) |
| 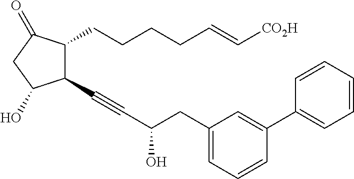 | (16) |

In one embodiment, the EP4 agonist is beraprost (1), nileprost (2), a tetrazole analog of nileprost (3), enprostil (4), nocloprost (5), arbaprostil (6), CP-533,536 (7), rivenprost (8), ONO-AE-1329 (9), AS-02 (10), AGN-205203 (11), L-902688 (12), eptaloprost (13), ONO-8815Ly (14), ciprosten (15), or FTA-2062, which is named (2E)-17,18,19,20-tetranor-16-(3-biphenyl)-2,3,13,14-tetradehydro-PGE 1 (16), also known as EP4RAG. In a specific embodiment, the EP4 receptor agonist is beraprost sodium, which is compound (1), named (+)-[1R,2R,3aS,8bS]-2,3,3a,8b-tetrahydro-2-hydroxyl-1-[(E)-(3S,4RS)-3-hydroxyl-4-methyl-1-octen-6-ynyl)-1H-cyclopenta[b]benzofuran-5-butanoic acid, sodium salt.

### Antiviral Agents

In one embodiment, the antiviral agent is a neuroaminidase inhibitor. Suitable neuroaminidase inhibitors include, but are not limited to, oseltamivir (Tamiflu® a trade name by Genentech of South San Francisco, Calif., USA), zanamivir (Relenza® a trade name by GlaxoSmithKline of Brentford, London, UK) and peramivir (produced by BioCryst Pharmaceuticals Inc. of Birmingham, Ala.). In one embodiment, the antiviral agent is an M2 channel inhibitor, for example, but not limited to, amantadine and rimantadine. In another embodiment, the antiviral agent is a polymerase inhibitor, such as, but not limited to, inhibitor T-705 (favipiravir, 6-fluoro-3-hydroxy-2-pyrazinecarboxamide, produced by Toyama Chemical Co., Ltd. of Toyama, Japan).

### Modes of Administration

The compounds may be administered to the patient in combination or adjunctively. The EP4 receptor agonist and the antiviral agent can be administered at the same time or sequentially. They may be separate formulations, or they may be combined and delivered as a single formulation. The dose may be given as a single dose to be administered once or divided into two or more daily doses. In one embodiment, the EP4 agonist and the antiviral are each administered twice daily, in another embodiment, each are administered once daily.

The individual amounts of the EP4 agonist and the antiviral agent that will be “effective” will be an amount that would be optimal or suboptimal if the EP4 receptor agonist or the antiviral agent were used alone (that is, not in combination) to treat the same viral disease. The effective amount of active ingredient may vary depending on the route of administration, the age and weight of the patient, the nature and severity of the disorder to be treated, and similar factors. The effective amount can be determined by methods known to those of skill in the art. The EP4 receptor agonists and antiviral agents enumerated herein are typically well studied and have dosing regimens for humans.

Generally, the EP4 receptor agonist is administered with the antiviral. The EP4 receptor agonist and the antiviral agent may be administered at optimal dosages for individual treatments, or one or both of the agonist and the antiviral can be dosed at a level that would be suboptimal if administered individually for a given patient. In one embodiment, at least one of the EP4 receptor agonist and the antiviral is at or below the optimal dosage for the given patient. In one embodiment, both the EP4 receptor agonist and the antiviral agent are administered at suboptimal dosage.

In one embodiment, the EP4 receptor agonist is administered at optimal dosage and the antiviral is administered at suboptimal dosage, where the suboptimal dosage is between about 10% and 80% of the optimal dosage for a given patient. In one embodiment, the EP4 receptor agonist is administered at optimal dosage and the antiviral is administered at suboptimal dosage, where the suboptimal dosage is between about 15% and 50% of the optimal dosage for a given patient. In one embodiment, the EP4 receptor agonist is administered at optimal dosage and the antiviral is administered at suboptimal dosage, where the suboptimal dosage is between about 20% and 50% of the optimal dosage for a given patient.

In one embodiment, the antiviral agent is administered at optimal dosage and the EP4 receptor agonist is administered at suboptimal dosage, where the suboptimal dosage is between about 10% and 100% of the median of the range of optimal dosage for a given patient. In one embodiment, the antiviral agent is administered at optimal dosage and the EP4 receptor agonist is administered at suboptimal dosage, where the suboptimal dosage is between about 30% and 80% of the median of the range of optimal dosage for a given patient. In one embodiment, the antiviral agent is administered at optimal dosage and the EP4 receptor agonist is administered at suboptimal dosage, where the suboptimal dosage is between about 30% and 50% of the median of the range of optimal dosage for a given patient.

In one embodiment, the EP4 receptor agonist is administered at suboptimal dosage and the antiviral agent is also administered at suboptimal dosage for a given patient. In one embodiment, suboptimal dosage for the EP4 receptor agonist is between about 10% and 100% of the median of the range of optimal dosage for a given patient, in another embodiment between about 30% and 80% of the median of the range of optimal dosage for a given patient, in another embodiment between about 30% and 50% of the median of the range of optimal dosage for a given patient. In one embodiment, suboptimal dosage for the antiviral agent is between about 10% and 80% of the optimal dosage for a given patient, in another embodiment between about 15% and 50% of the optimal dosage for a given patient, in yet another embodiment between about 20% and 50% of the optimal dosage for a given patient.

By way of a non-limiting example, for the EP4 receptor agonist, beraprost, a current therapy for optimal dosage is 20 mcg to 60 mcg up to 3 times a day, that is between 20 mcg and 180 mcg of beraprost per day, depending on a given patient. The median optimal dosage is 100 mcg per day. In one embodiment, a suboptimal dose of beraprost is between about 10 mcg and about 100 mcg per day, in another embodiment between about 30 mcg and about 80 mcg per day, in another embodiment between about 30 mcg and about 50 mcg per day. An optimal dose of the antiviral oseltamivir, Tamiflu®, is twice daily for 5 days of either 75 mg for adults or 30 mg for children, that is, 150 mg per day for an adult and 60 mg per day for a child. In one embodiment, a suboptimal dose of oseltamivir is between about 15 mg and about 120 mg per day for an adult, and between about 6 mg and about 48 mg per day for a child. In one embodiment, a suboptimal dose of oseltamivir is between about 23 mg and about 75 mg per day for an adult, and between about 9 mg and about 30 mg per day for a child. In another embodiment, a suboptimal dose of oseltamivir is between about 30 mg and about 75 mg per day for an adult, and between about 12 mg and about 30 mg per day for a child. In a specific embodiment, 20 mcg twice a day of beraprost is coadministered with either 30 mg of oseltamivir once a day for adults, or 15 mg of oseltamivir once a day for children.

In one embodiment, an anti-inflammatory compound may also be coadministered with the EP4 receptor agonist and the antiviral agent. Suitable anti-inflammatory compounds include, for example, non-steroidal anti-inflammatory agents (NSAID's) as well as steroidal anti-inflammatory agents. Suitable NSAID's include, but are not limited to ibuprofen, naproxen, fenoprofen, ketoprofen, flurbiprofen, oxaprozin, indomethacin, sulindac, etodolac, ketorolac, diclofenac, nabumetone, piroxicam, meloxicam, tenoxicam, droxicam, lornoxicam, isoxicam, mefenamic acid, meclofenamic acid, flufenamic acid, tolfenamic acid and celecoxib. Suitable steroidal anti-inflammatory agents include, but are not limited to, corticosteroids such as synthetic glucocorticoids. Routes of administration of NSAID's are typically oral, and steroids can be taken, for example, orally, inhaled, injected and the like.

In one embodiment, one or more NSAID's are coadministered with the EP4 receptor agonist and the antiviral agent. In one embodiment, a single NSAID is coadministered with the EP4 receptor agonist and the antiviral agent. In one embodiment, the NSAID is ibuprofen.

In one embodiment, an analgesic compound may also be coadministered with the EP4 receptor agonist and the antiviral agent. In one embodiment the analgesic is acetaminophen (paracetamol).

Immune response modulators which have distinct mechanism of action may also be coadministered with the EP4 receptor agonist and the antiviral agent. Immune response modulators may be used, for example, to modify the immune system during viral infection. In one embodiment, the immune response modulator is AAL-R, which modulates the immune response by interacting with the spingosine 1-phosphate (SIP) receptor as an agonist. The chemical name for AAL-R is (R)-2-amino-4-(4-heptyloxyphenyl)-2-methylbutanol and is described in, for example, Marsolais Mol Pharmaol 2008; 74:896-903, which is incorporated by reference herein for all purposes.

A nuclear receptor peroxisome proliferator-activated receptor gamma (PPAR-γ) agonist may also be co-administered with the EP4 receptor agonist and the antiviral agent. The biologic effect of a PPAR-γ agonist is partially mediated by modulating the immune response through a distinctly different signaling pathway than an EP4 agonist. In one embodiment, the PPAR-γ agonist is pioglitazone or rosiglitazone.

### Pharmaceutical Compositions

Pharmaceutical compositions for use according to embodiments described herein comprise the EP4 receptor agonist and the antiviral agent, either together in a single dosage form or in separate dosage forms in an effective amount (that is, an amount effective to treat an influenza viral disease or a SARS disease synergistically when applied together as a combination therapy) and one or more pharmaceutically acceptable excipients. Pharmaceutical compositions may also include one or more anti-inflammatory agents, and/or analgesics, PPAR-γ agonists and immune response modulators.

Suitable excipients may include, but are not limited to, pharmaceutical, organic or inorganic inert carrier materials suitable for enteral, parenteral or topical administration which do not deleteriously react with the active compounds. Suitable pharmaceutically acceptable carriers include, but are not limited to, water, salt solutions, alcohols, gelatine, gum arabic, lactate, starch, magnesium stearate, talc, vegetable oils, polyalkylene glycols, polyvinyl pyrrolidone, hydroxyl-methylcellulose, silicic acid, viscous paraffin, fatty acid monoglycerides and diglycerides, and the like. The pharmaceutical products may be in solid form, for example as tablets, coated tablets, suppositories or capsules, or in liquid form, for example as solutions, suspensions or emulsions. They may additionally comprise, where appropriate, auxiliary agents such as lubricants, preservatives, stabilizers, wetting agents, emulsifiers, salts to alter the osmotic pressure, buffers, coloring, flavoring, and/or aromatic substances and the like that do not deleteriously react with the active compounds.

Examples of suitable pharmaceutical compositions include aerosol solutions, aerosol powders, tablets, capsules, sterile injectable solutions and the like, as would be appreciated by one of ordinary skill in the art. Aerosol solutions are expediently produced for delivery via inhalation. Particularly suitable for oral use are tablets, coated tablets or capsules with talc and/or carbohydrate carriers or binders, such as, for example, lactose, maize starch or potato starch. Use is also possible in liquid form, such as, for example, as fluid to which a sweetener is added where appropriate. A controlled release formulation for beraprost has been patented. Sterile, injectable, aqueous or oily solutions are used for parenteral administration, as well as suspensions, emulsions or implants, including suppositories. Ampoules are convenient unit dosages. Sustained release compositions can be formulated including those wherein the active compound is protected with differentially degradable coatings, for example, by microencapsulation, multiple coatings, etc. Carrier systems which can also be used are surface-active excipients such as salts of bile acids or animal or vegetable phospholipids, but also mixtures thereof, and liposomes or constituents thereof. Transdermal patches may also be used as delivery means.

One embodiment is a pharmaceutical composition for treating a viral disease, including an antiviral agent in combination with an EP4 receptor agonist. In one embodiment, at least one of the EP4 receptor agonist and the antiviral agent is present at a suboptimal dosage. In one embodiment, the EP4 receptor agonist is present at between 10% and 100% of the median of the range of optimal dosage for a given patient. In one embodiment, the antiviral agent is present in the composition at between 10% and 80% of the optimal dosage for a given patient, in another embodiment at between 15% and 50% of the optimal dosage for the given patient. In one embodiment the viral disease is caused by an influenza virus, in another embodiment by a corona virus. In one embodiment, the pharmaceutical composition is formulated for administration to a human patient. In one embodiment, the antiviral agent is selected from a viral protein M2 ion channel inhibitor, a neuraminidase inhibitor, an RNA replication and translation inhibitor and a polymerase inhibitor, in another embodiment the antiviral agent is amantadine or rimantadine. In one embodiment, the antiviral agent is oseltamivir, zanamivir, peramivir or {(4S,5R,6R)-5-acetamido-4-guanidino-6-[(1R,2R)-2-hydroxy-1-methoxy-3-(octanoyloxy)propyl]-5,6-dihydro-4H-pyran-2-carboxylic acid, in another embodiment the antiviral agent is ribavirin or 6-fluoro-3-hydroxy-2-pyrazinecarboxamide. In one embodiment the EP4 receptor agonist is beraprost sodium, (+)-[1R,2R,3aS,8bS]-2,3,3a,8b-tetrahydro-2-hydroxyl-1-[(E)-(3S)-3-hydroxyl-4-methyl-1-octen-6-ynyl)-1H-cyclopenta[b]benzofuran-5-butanoic acid, sodium salt, (+)-[1R,2R,3aS,8bS]-2,3,3a,8b-tetrahydro-2-hydroxyl-1-[(E)-(3S, The method of claim 1, further including coadministering (R)-2-amino-4-(4-heptyloxyphenyl)-2-methylbutanol. In another embodiment, the EP4 agonist is 4R)-3-hydroxyl-4-methyl-1-octen-6-ynyl)-1H-cyclopenta[b]benzofuran-5-butanoic acid, sodium salt, (+)-[1R,2R,3aS,8bS]-2,3,3a,8b-tetrahydro-2-hydroxyl-1-[(E)-(3S,4S)-3-hydroxyl-4-methyl-1-octen-6-ynyl)-1H-cyclopenta[b]benzofuran-5-butanoic acid, sodium salt or (+)-[1R,2R,3aS,8bS]-2,3,3a,8b-tetrahydro-2-hydroxyl-1-[(E)-(3S)-3-hydroxyl-4-methyl-1-octen-6-ynyl)-1H-cyclopenta[b]benzofuran-5-butanoic acid, sodium salt. In another embodiment, the EP4 receptor agonist is nileprost, (E)-5-cyano-5-[(1S,5R,6R,7R)-7-hydroxy-6-[(E)-(3S,4RS)-3-hydroxy-4-methyl-1-octenyl]-2-oxa-bicyclo[3.3.0]octan-3-yliden]pentanoic acid and isomers (E)-5-cyano-5-[(1S,5R,6R,7R)-7-hydroxy-6-[(E)-(3S,4S)-3-hydroxy-4-methyl-1-octenyl]-2-oxa-bicyclo[3.3.0]octan-3-yliden]pentanoic acid or (E)-5-cyano-5-[(1S,5R,6R,7R)-7-hydroxy-6-[(E)-(3S,4R)-3-hydroxy-4-methyl-1-octenyl]-2-oxa-bicyclo[3.3.0]octan-3-yliden]pentanoic acid or (E)-[(3aR,4R,5R,6aS)-hexahydro-5-hydroxy-4-[(1E,3S)-3-hydroxy-4-methyl-1-octenyl]-2H-cyclopenta[b]furan-2-ylidene]-1H-tetrazole-5-2E-pentanenitrile. In yet another embodiment, the EP4 receptor agonist in the pharmaceutical composition is

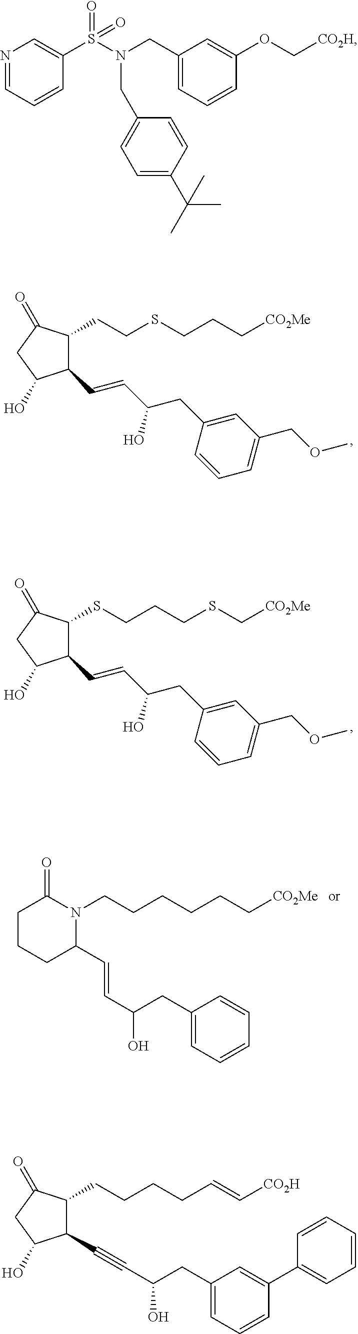

In one embodiment, the pharmaceutical composition is further characterized by being used such that it is coadministered with pioglitazone or rosiglitazone. In one embodiment, the pharmaceutical composition is further characterized by being used such that it is administered either in combination or adjunctively with an anti-inflammatory agent. In one embodiment, the anti-inflammatory agent is an NSAID, in another embodiment, the anti-inflammatory agent is a steroid. In one embodiment, the pharmaceutical composition including an EP4 receptor agonist and an antiviral agent, is in a single dosage form, with one or more pharmaceutically acceptable excipients.

The following specific embodiments are to be construed as merely illustrative, and therefore not limiting.

### EXAMPLES

### Example 1

Compounds: Beraprost, oseltamivir and ribavirin were prepared in physiological salt saline solution (PSS) for animal studies. Beraprost solution was prepared by dissolving 6.3 mg of beraprost in 58 mL of PSS and diluted as needed. An oseltamivir solution of 0.9 mg/mL was diluted into water. Ribavirin solution was prepared by dissolving 217.5 mg into 29 mL of water. A volume of 0.1 mL was used for ip and po administration.

Virus: Influenza A/Duck/MN/1525/81 (H5N1) virus was obtained from Dr. Robert Webster of St. Jude Hospital, Memphis, Tenn. It was passaged through mice until adapted to the point of being capable of inducing pneumonia-associated death in the animals.

Animals: Female 18-20 g BALB/c mice were obtained from Charles River Laboratories (Wilmington, Mass.) for this study. They were maintained on Wayne Lab Blox and tap water ad libitum. They were quarantined for 24 h prior to use.

Studies of pathogenicity of A/Duck/MN/1525/81 (H5N1) virus in mice: Female 18-20 g BALB/c mice from Charles River Laboratory were dosed with PSS (ip, bid), beraprost (ip), oseltamivir (os), ribavirin (ip) and combination of beraprost and oseltamivir for 5 to 10 days beginning on day 0 of viral dose. The animals were dosed at 8 am and 4 pm. An LD100 viral dose [about 1×10⁵ TCID50 (1:400)] of Influenza A/Duck/Mn/1525/81 (H5N1) virus was administered intranasally. Mice were individually weighed prior to treatment and then every day thereafter to assess the effects of each treatment on ameliorating weight loss due to virus infection. On selected days, five mice from each treatment group and 10 mice from the placebo group were sacrificed when possible, and the lungs were scored for consolidation and discoloration, and then homogenized and titrated for the presence of virus. Surviving mice from each group were followed for death up to day 21. Toxicity controls were run in parallel using 3 animals per group.

Effect of beraprost or oseltamivir treatment on lethal influenza infections in mice. In the experiment with dosing from viral infection through day 10, 1 out of 10 mice survived in the PSS group, 6 of 10 mice in the beraprost (1.2 mg/Kg/day), and 8 out of 10 in the oseltamivir (5 mg/Kg/day) group survived to day 21. All mice survived in combination treatment group, beraprost (1.2 mg/kg/da) and oseltamivir (5 mg/kg/da). The mean day of death (MDD) was significantly higher in the combination group, >21 days, compared to the PSS, beraprost and oseltamivir treated groups, 7.4±1.2, 9.0±3.9, and 9.0±0.0 respectively.

Similar survival rates which were not statistically different from each other were observed with a lower dose of oseltamivir (1 mg/Kg/da, 70%), PSS (20%) and beraprost (1.2 mg/kg/da, 50%) with MDD values of 11.4±1.8, 8.4±3.2, and 8.0±2.0 respectively. Combination therapy of oseltamivir (1 mg/Kg/da) and beraprost (1.2 mg/kg/da) resulted in survival of all mice and an extension of MDD to >21 days was significantly differ from controls and monotherapies.

The improvement in lung function coincided with an amelioration of actual pathogenesis observed in mice receiving the various treatments compared to that observed in untreated mice. Thin sections of lungs from each group of mice were characterized for pathology by a board-certified pathologist. For all treatments groups at day 6, including the mice treated with placebo, the lungs were typically characterized by scattered bronchioles segmentally lined with necrotic epithelial cells, and the bronchioles contained luminal cellular debris. Surrounding alveoli contained moderate numbers of neutrophils and macrophages. However, in three of five mice receiving the combination of beraprost and either dose of oseltamivir, the infiltration by macrophages and neutrophils was described as small, not moderate.

Effect of ribavirin treatment on lethal influenza infections in mice. All the mice in the group treated with ribavirin (75 mg/kg/d) were significantly protected against death (100%, P<0.001) at both the 5 and 10 day dosing regimen.

### Example 2

Animals: Female 18-20 g BALB/c mice were obtained from Charles River Laboratories. The mice were quarantined for 72 hours before use and maintained on Teklad Rodent Diet (Harlan Teklad) and tap water at the Laboratory Animal Research Center of Utah State University.

Virus: Influenza A/NWS/33 (H1N1) was initially provided by Dr. Kenneth Cochran (University of Michigan, Ann Arbor). The virus was passaged 9 times in MDCK cells, and a pool was prepared and pre-titrated for lethality in mice.

Compounds: The compound was prepared in PBS for administration as described above.

Experimental design: Animal numbers and study groups are described in Table 2. Mice were anesthetized by i.p. injection of ketamine/xylazine prior to challenge by the intranasal route with a 90 μl suspension of influenza virus. Monotherapy treatment groups consisted of oseltamivir administered twice a day by the oral route at 0.05 or 0.1 mg/kg/day, and beraprost administered twice a day by the intraperitoneal route at 1.2 mg/kg/day. All oseltamivir and beraprost groups received treatment for 10 days beginning 4 hours prior to virus exposure. Drug combination treatment consisted of oseltamivir at 0.05 or 0.1 mg/kg/day combined with beraprost at 1.2 mg/kg/day. Ribavirin was administered twice a day by the intraperitoneal route at 75 mg/kg/day for 5 days beginning 4 hours prior to virus exposure. All treatments were administered 12 hours apart. Following infection, the mice were observed for 21 days.

TABLE 2

| No./ | Infected? | | | Treatment | Obser- |
|---|---|---|---|---|---|
| Cage | (Y or N) | Compound | Dosage | Schedule | vations |
| 20 | Y | Placebo | — | bid X 10, 12 h | Observed |
| | | | | apart, beg day 0, | for weight |
| | | | | 4 hr prior to | loss and |
| | | | | infection | death |
| 10 | Y | Oseltamivir | 0.1 | bid X 10, 12 h | through |
| | | | mg/kg/day | apart, beg day 0, | day 21 |
| | | | | 4 hr prior to | |
| | | | | infection | |
| 10 | Y | Oseltamivir | 0.05 | bid X 10, 12 h | |
| | | | mg/kg/day | apart, beg day 0, | |
| | | | | 4 hr prior to | |
| | | | | infection | |
| 10 | Y | beraprost | 1.2 | bid X 10, 12 h | |
| | | | mg/kg/day | apart, beg day 0, | |
| | | | | 4 hr prior to | |
| | | | | infection | |
| 10 | Y | Oseltamivir + | 0.1 | bid X 10, 12 h | |
| | | beraprost | mg/kg/ | apart, beg day 0, | |
| | | | day + 1.2 | 4 hr prior to | |
| | | | mg/kg/day | infection | |
| 10 | Y | Oseltamivir + | 0.05 | bid X 10, 12 h | |
| | | beraprost | mg/kg/ | apart, beg day 0, | |
| | | | day + 1.2 | 4 hr prior to | |
| | | | mg/kg/day | infection | |
| 10 | Y | Ribavirin | 75 | bid X 5, 12 h | |
| | | | mg/kg/day | apart, beg day 0, | |
| | | | | 4 hr prior to | |
| | | | | infection | |
| 10 | N | None | — | — | Observed |
| | | | | | for weight |
| | | | | | gain |

Arterial oxygen saturation (SaO₂) determinations: SaO₂ measurements were made using the MouseOx™ (STARR Life Sciences, Pittsburgh, Pa.) pulse oximeter with collar attachment designed specifically to measure SaO₂ levels in rodents. SaO₂ levels were measured on days 5, 6, 7, and 8 after virus exposure, since on these days most animals show the most severe clinical signs and/or die. Mean SaO₂ levels were determined on each date for each treatment group and analyzed for significant differences between treatment groups by the Kruskal-Wallis test, followed by Dunn's post test for evaluating significant pairwise comparisons.
Additional statistical analyses: Kaplan-Meier survival curves were generated and compared by the Log-rank (Mantel-Cox) test followed by pairwise comparison using the Gehan-Breslow-Wilcoxon test in Prism 5.0b (GraphPad Software Inc.). The mean body weights were analyzed by one-way ANOVA followed by Tukey's multiple comparison tests using Prism 5.0b.
Effects of combination therapy with dosing 4 hours before infection on Survival of Mice: In the control groups, all animals survived in the ribavirin group and no mice survived in the PSS group. In the monotherapy groups (two doses of oseltamivir or one dose of beraprost), no animals survived. In the combination group of beraprost and oseltamivir (0.05 mg/Kg/day), one of ten animals survived. In the combination group of beraprost+oseltamivir (0.1 mg/kg/day), eight of ten animals survived.

### Example 3

Animals: Female 18-20 g BALB/c mice were obtained from Charles River Laboratories. The mice were quarantined for 72 hours before use and maintained on Teklad Rodent Diet (Harlan Teklad) and tap water at the Laboratory Animal Research Center of Utah State University.

Virus: Influenza A/NWS/33 (H1N1) was initially provided by Dr. Kenneth Cochran (University of Michigan, Ann Arbor). The virus was passaged 9 times in MDCK cells, and a pool was prepared and pre-titrated for lethality in mice.

Compounds: The compound was prepared in PBS for administration.

Experimental design: Animal numbers and study groups are described in Table 3. Mice were anesthetized by i.p. injection of ketamine/xylazine prior to challenge by the intranasal route with a 90 μl suspension of influenza virus. Monotherapy treatment groups consisted of oseltamivir administered twice a day by the oral route at 0.05 or 0.1 mg/kg/day, and beraprost administered twice a day by the intraperitoneal route at 1.2 mg/kg/day. All oseltamivir and beraprost groups received treatment for 10 days beginning 24 hours after virus exposure to mice. Drug combination treatment consisted of oseltamivir at 0.05 or 0.1 mg/kg/day combined with beraprost at 1.2 mg/kg/day. Ribavirin was administered twice a day by the intraperitoneal route at 75 mg/kg/day for 5 days beginning 4 hours prior to virus exposure. All treatments were administered 12 hours apart. Following infection the mice were observed for 21 days.

TABLE 3

| No./ | Infected? | | | Treatment | Obser- |
|---|---|---|---|---|---|
| Cage | (Y or N) | Compound | Dosage | Schedule | vations |
| 20 | Y | Placebo | — | bid X 10, 12 h | Observed |
| | | | | apart, beg day 0, | for weight |
| | | | | 4 hr prior to | loss and |
| | | | | infection | death |
| 10 | Y | Oseltamivir | 0.1 | bid X 10, 12 h | through |
| | | | mg/kg/day | apart, beg 24 hr | day 21 |
| | | | | post-infection | |
| 10 | Y | beraprost | 1.2 | bid X 10, 12 h | |
| | | | mg/kg/day | apart, beg 24 hr | |
| | | | | post-infection | |
| 10 | Yes | Oseltamivir + | 0.1 | bid X 10, 12 h | |
| | | beraprost | mg/kg/ | apart, beg 24 hr | |
| | | | day + 1.2 | post-infection | |
| | | | mg/kg/day | | |
| 10 | Yes | Ribavirin | 75 | bid X 5, 12 h | |
| | | | mg/kg/day | apart, beg day 0, | |
| | | | | 4 hr prior to | |
| | | | | infection | |
| 10 | No | None | — | — | Observed |
| | | | | | for weight |
| | | | | | gain |

Arterial oxygen saturation (SaO₂) determinations: SaO₂ measurements were made using the MouseOx™ (STARR Life Sciences, Pittsburgh, Pa.) pulse oximeter with collar attachment designed specifically to measure SaO₂ levels in rodents. SaO₂ levels were measured on days 5, 6, 7, and 8 after virus exposure, since on these days most animals show the most severe clinical signs and/or die. Mean SaO₂ levels were determined on each date for each treatment group and analyzed for significant differences between treatment groups by the Kruskal-Wallis test, followed by Dunn's post test for evaluating significant pairwise comparisons.
Additional statistical analyses: Kaplan-Meier survival curves were generated and compared by the Log-rank (Mantel-Cox) test followed by pairwise comparison using the Gehan-Breslow-Wilcoxon test in Prism 5.0b (GraphPad Software Inc.). The mean body weights were analyzed by one-way ANOVA followed by Tukey's multiple comparison tests using Prism 5.0b.
Effects of combination therapy with dosing 24 hours after infection on Survival of Mice: Survival curves for groups with treatment beginning 24 hours after virus exposure showed that all 10 animals in the positive control group treated with ribavirin survived and none the animals in the negative control or PSS-treatment group survived. All animals in the monotherapy groups died after either oseltamivir or beraprost treatment. In the combination group of beraprost (1.2 mg/kg/day)+oseltamivir (0.1 mg/kg/day), eight of ten animals survived. In addition, a multiple comparison test of mean body weights for study groups with treatment beginning 24 hours after virus exposure showed that the beraprost (1.2 mg/kg/day)+oseltamivir (0.1 mg/kg/day) treatment allowed the mice to recover and gain weight more rapidly than monotherapy treatments alone. By days 18 and 20 post-infection, no significant difference could be observed between the ribavirin and the beraprost (1.2 mg/kg/day)+oseltamivir (0.1 mg/kg/day) treated groups. A comparison to the placebo group could not be completed for these dates because mice in the placebo group did not survive past day 10. Results of SaO₂ levels for study groups with beraprost, either alone or in combination were similar to uninfected animals with a significant difference (P<0.05) from placebo. Results of SaO₂ for oseltamivir monotherapy group were similar to placebo group (PSS treated).

This study demonstrates that twice-a-day treatment with a combination of beraprost at 1.2 mg/kg/day and oseltamivir at 0.1 mg/kg/day for ten days can improve the survival of mice following intranasal infection with influenza A/NWS/33 (H1N1) virus. These results were observed for mice beginning treatment 24 hours after challenge exposure. Mice receiving the combination therapy beginning 24 hours after challenge infection were able to recover more rapidly, as indicated by weight gain, than mice receiving monotherapy treatment alone. Furthermore, arterial oxygen saturation (SaO₂) levels were significantly higher following combination therapy beginning 24 hours after challenge exposure.

Although the foregoing invention has been described in some detail to facilitate understanding, the described embodiments are to be considered illustrative and not limiting. It will be apparent to one of ordinary skill in the art that certain changes and modifications can be practiced within the scope of the appended claims.

What is claimed is:

1. A method for treating a viral disease, the method comprising administering to a patient in need thereof an antiviral agent in combination with an EP4 receptor agonist.

2. The method of claim 1, wherein at least one of the EP4 receptor agonist and the antiviral agent is administered at a suboptimal dosage.

3. The method of claim 2, wherein the EP4 receptor agonist is administered at between about 10% and about 100% of the median of the range of optimal dosage for a given patient.

4. The method of claim 2, wherein the antiviral agent is administered at between about 10% and about 80% of the optimal dosage for a given patient.

5. The method of claim 3, wherein the antiviral agent is administered at between about 15% and about 50% of the optimal dosage for the given patient.

6. The method of claim 1, wherein the viral disease is caused by an influenza virus.

7. The method of claim 1, wherein the viral disease is caused by a corona virus.

8. The method of claim 1, wherein the patient is a human.

9. The method of claim 1, wherein the antiviral agent is selected from a viral protein M2 ion channel inhibitor, a neuraminidase inhibitor, an RNA replication and translation inhibitor and a polymerase inhibitor.

10. The method of claim 1, wherein the antiviral agent is amantadine or rimantadine.

11. The method of claim 1, wherein the antiviral agent is oseltamivir, zanamivir, peramivir or {(4S,5R,6R)-5-acetamido-4-guanidino-6-R1R,2R)-2-hydroxy-1-methoxy-3-(octanoyloxy)propyl]-5,6-dihydro-4H-pyran-2-carboxylic acid.

12. The method of claim 1, wherein the antiviral agent is ribavirin.

13. The method of claim 1, wherein the antiviral agent is 6-fluoro-3-hydroxy-2-pyrazinecarboxamide.

14. The method of claim 1, wherein the EP4 receptor agonist is beraprost sodium, (+)-[1R,2R,3aS,8bS]-2,3,3a,8b-tetrahydro-2-hydroxyl-1-[(E)-(3S)-3-hydroxyl-4-methyl-1-octen-6-ynyl)-1H-cyclopenta[b]benzofuran-5-butanoic acid, sodium salt, (+)-[1R,2R,3aS,8bS]-2,3,3a,8b-tetrahydro-2-hydroxyl-1-[(E)-(3S, The method of claim 1, further including coadministering (R)-2-amino-4-(4-heptyloxyphenyl)-2-methylbutanol.

15. The method of claim 1, further including coadministering pioglitazone or rosiglitazone.

16. The method of claim 1, wherein the EP4 agonist is 4R)-3-hydroxyl-4-methyl-1-octen-6-ynyl)-1H-cyclopenta[b]benzofuran-5-butanoic acid, sodium salt, (+)-[1R,2R,3aS,8bS]-2,3,3a,8b-tetrahydro-2-hydroxyl-1-[(E)-(3S,4S)-3-hydroxyl-4-methyl-1-octen-6-ynyl)-1H-cyclopenta[b]benzofuran-5-butanoic acid, sodium salt or (+)-[1R,2R,3aS,8bS]-2,3,3a,8b-tetrahydro-2-hydroxyl-1-[(E)-(3S)-3-hydroxyl-4-methyl-1-octen-6-ynyl)-1H-cyclopenta[b]benzofuran-5-butanoic acid, sodium salt.

17. The method of claim 1, wherein the EP4 receptor agonist is nileprost, (E)-5-cyano-5-[(1S,5R,6R,7R)-7-hydroxy-6-[(E)-(3S,4RS)-3-hydroxy-4-methyl-1-octenyl]-2-oxa-bicyclo[3.3.0]octan-3-yliden]pentanoic acid and isomers (E)-5-cyano-5-[(1S,5R,6R,7R)-7-hydroxy-6-[(E)-(3S,4S)-3-hydroxy-4-methyl-1-octenyl]-2-oxa-bicyclo[3.3.0]octan-3-yliden]pentanoic acid or (E)-5-cyano-5-[(1S,5R,6R,7R)-7-hydroxy-6-[(E)-(3S,4R)-3-hydroxy-4-methyl-1-octenyl]-2-oxa-bicyclo[3.3.0]octan-3-yliden]pentanoic acid or (E)-[(3aR,4R,5R,6aS)-hexahydro-5-hydroxy-4-[(1E,3S)-3-hydroxy-4-methyl-1-octenyl]-2H-cyclopenta[b]furan-2-ylidene]-1H-tetrazole-5-2E-pentanenitrile.

18. The method of claim 1, wherein the EP4 receptor agonist is

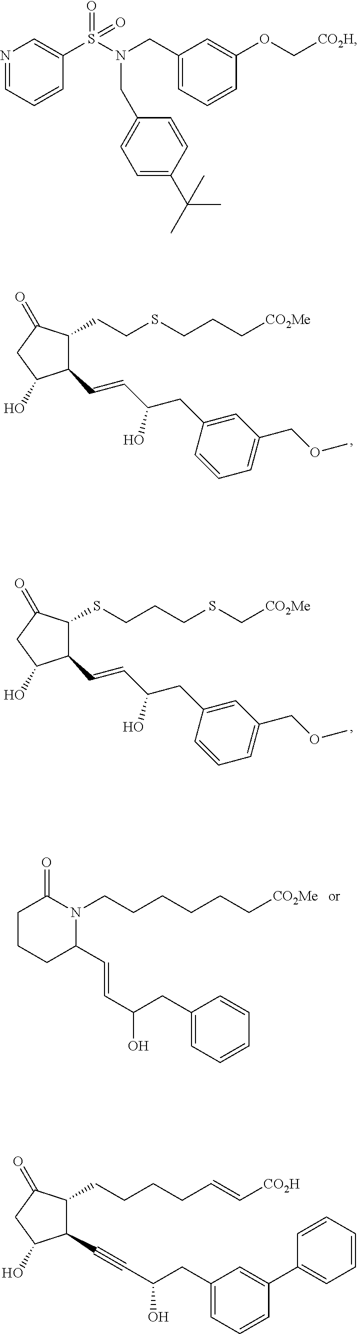

19. The method of claim 1, wherein the antiviral agent and the EP4 receptor agonist are administered either in combination or adjunctively with an anti-inflammatory agent.

20. The method of claim 19, wherein the anti-inflammatory agent is an NSAID.

21. The method of claim 19, wherein the anti-inflammatory agent is a steroid.

22. A pharmaceutical composition comprising an EP4 receptor agonist and an antiviral agent, in a single dosage form, with one or more pharmaceutically acceptable excipients.

23. A kit comprising a pharmaceutical formulation comprising an antiviral agent and an EP4 receptor agonist.

24. A kit comprising a first pharmaceutical formulation comprising an antiviral agent and a second pharmaceutical formulation comprising an EP4 receptor agonist.

25. A combination of an EP4 receptor agonist and an antiviral for use in the treatment of an infection in a human by an influenza virus and/or a corona virus.
